(19) United States
(12) Patent Application Publication
(10) Pub. No.: US 2007/0191632 A1
(43) Pub. Date: Aug. 16, 2007

(54) Production of compounds with chf2- or chf groups

(75) Inventor: Max Braun, Wedemark (DE)

Correspondence Address: CONNOLLY BOVE LODGE & HUTZ, LLP, P O BOX 2207, WILMINGTON, DE, 19899, US

(73) Assignee: Solvay Organics Gmbh, Hannover (DE)

(21) Appl. No.: 10/591,877

(22) PCT Filed: Feb. 4, 2005
(86) PCT No.: PCT/EP05/01123

§ 371 Date: Oct. 10, 2006

(30) Foreign Application Priority Data
Mar. 8, 2004 (EP) 04005507.1

(51) Int. Cl. C07C 69/63; C07C 45/61

(52) U.S. Cl. 560/227; 568/458

(57) ABSTRACT

Compounds having a CHF₂C(O) or CHFC(O) group can be prepared from the corresponding compounds having a CClF₂C(O) or CClFC(O) group or the corresponding bromine or iodine compound and zinc in the presence of an alcohol as a proton source. Preference is given to preparing monoesters having one CHF₂C(O) group or diesters having two CHFC(O) groups or (O)CCHFC(O) groups in this way.

The invention relates to a process for preparing compounds having a CHF₂C(O) or CHFC(O) group by hydrodehalogenation, preferably hydrodechlorination, from corresponding bromine, iodine or chlorine compounds, preferably from compounds having a CClF₂C(O) or CClFC(O) group.

Compounds having a CHF₂C(O) or CHFC(O) group, especially ester compounds and diester compounds, are valuable intermediates in chemical synthesis.

The preparation of such compounds from corresponding chlorine compounds and exchange of the chlorine atom for hydrogen is already known. Takashi Tsukamoto and Tomoya Kitazume state, in J. Chem. Soc. Perkin Trans. 1993, pages 1177 to 1181, that ethyl chlorodifluoroacetate with zinc in dimethylformamide and subsequent acidic hydrolysis gives rise quantitatively to ethyl difluoroacetate (see page 1177, left-hand column). However, it is not stated how the ester might be isolated.

A. Yakubovich and S. M. Rozensthein show, in Journal of General Chemistry USSR, Volume 31 (1961), pages 1866 to 1870, that there is exchange of the chlorine atom in esters substituted in the α- and β-position by chlorine and/or bromine for hydrogen, and addition of an alkoxy group.

It is an object of the present invention to specify an improved process for preparing compounds having a CHF₂C(O) or CHFC(O) group, particularly from corresponding ester or diester compounds. This object is achieved by the process according to the invention.

The process according to the invention envisages the preparation of compounds having a CFₙHC(O) group from a CFₙXC(O) group and zinc in the presence of an alcohol as a proton source, where n is 1 or 2 and X is bromine, iodine or preferably chlorine, by exchanging X for hydrogen, excluding compounds which are substituted by X both in the α-position and in the β-position. This also includes compounds having an (O)CCHFC(O) group, which are prepared from a compound having an (O)CCClFC(O) group. In the context of the present invention, X is exchanged for hydrogen; therefore, those compounds having two (or more) X substitutents for which there is both exchange of X for hydrogen and for an alkoxy radical (stems from alcohol present) are not encompassed by the invention. Of course, it is also possible to prepare compounds having two or more CFₙHC(O) groups from compounds having two or more CFₙXC(O) groups. In that case, correspondingly more zinc and alcohol is used.

The alcohol serves as the proton source for the reaction; it can also be used in excess and then also serves as the solvent. The particular product compound can also be added as a solvent. Dimethylformamide or other carboxamides are preferably not present in the reaction mixture.

A preferred embodiment of the process according to the invention is characterized in that compounds having one or two CFₙHC(O) groups are prepared from a compound having one or two CFₙXC(O) groups, where n and X are each as defined above. Preference is given to preparing compounds having one or two CFₙHC(O) groups from a compound having one or two CFₙClC(O) groups.

Particular preference is given to preparing an ester of the formula R¹CFHC(O)OR² in which R¹ is F; C1-C5-alkyl; or C1-C5-alkyl which is substituted by at least 1 fluorine atom; and R² is C1-C5-alkyl; or C1-C5-alkyl which is substituted by at least 1 fluorine atom. Preference is further given to preparing a diester of the formula R³OC(O)CFHC(O)OR³ in which R³ is C1-C5-alkyl; or C1-C5-alkyl which is substituted by at least 1 fluorine atom. Preferred starting compounds are the particular chlorine-substituted compounds.

R¹ is most preferably F or C1-C3 which is part-fluorinated or perfluorinated.

R² and R³ are preferably each methyl, ethyl, n-propyl or isopropyl.

R¹ is preferably F or CF₃.

The alcohol used as the proton source (optionally used in excess also as the solvent) corresponds appropriately to the R² or R³ radical.

In one embodiment, the ester is prepared in situ from the corresponding acid chloride and alcohol. In this case, the alcohol is not just the proton source, but also serves to esterify the acid chloride. Accordingly, more alcohol must be used. Since the alcohol is, though, appropriately used in excess as the solvent, this is not a problem.

It may be advantageous to perform the reaction in the presence of an aprotic solvent. In this case, the aprotic solvent used is preferably at least predominantly the product to be prepared, for example the ester or diester having one or more CHF₂C(O) or CHFC(O) groups. Nitriles are preferably not present as solvents. As already stated above, preference is given to not using carboxamides such as DMF as solvents.

Preference is given to using from 0.9 to 2.1 equivalents of zinc per chlorine atom to be exchanged. Preference is given to using from about 1.1 to 2 zinc atoms per chlorine atom; a stoichiometric excess of zinc has been found to be advantageous.

The temperature at which the reaction between bromine-, iodine- or chlorine-containing starting compound, zinc and alcohol is performed is advantageously between 50° C. and the boiling point of the appropriate alcohol.

The isolation can be effected by customary methods.

The invention further provides the azeotrope of methyl difluoroacetate and methanol, which can be distilled in the case of reaction of methyl chlorodifluoroacetate, zinc and methanol. This azeotrope is usable, for example, as a solvent or cleaning agent. The advantage is that it can be purified by redistillation without its composition changing. However, it can also be added in the reaction mixture of zinc and methyl chlorodifluoroacetate and, if appropriate, methanol. Depending on the amount of azeotrope added, the amount of methanol which serves as the proton source can be reduced; if appropriate, the methanol required as the proton source can be omitted entirely. In that case, the azeotrope serves as the solvent and, owing to the methanol present, as the proton source.

The process according to the invention has the advantage that high yields and high selectivities are achieved. Solvents such as DMF are also more difficult to dispose of.

The examples which follow are intended to further illustrate the invention without restricting its scope.

### EXAMPLES

### Example 1

### Preparation of Methyl Difluoroacetate from Methyl Chlorodifluoroacetate and Zinc

General procedure: starting compound, zinc and alcohol were reacted with one another in the specified proportions and during the reaction time and temperature specified. The distillative removal of the methyl ester always gave rise to a fraction of an azeotrope of methyl difluoroacetate and methanol with a constant boiling point of 64° C. at ambient pressure. This mixture was admixed with benzotrifluoride as an internal standard and the yield was confirmed by ¹H and ¹⁹F NMR spectra. The azeotrope can be reacted further to give other difluoroacetic acid compounds. NH₃ can be used to convert the ester, for example, in quantitative yield to difluoroacetamide.

Reaction conditions are compiled in Table 1.

Abbreviations:

MeCDFA=methyl chlorodifluoroacetate

EtCDFA=ethyl chlorodifluoro

CDFACl=chlorodifluoroacetyl chloride

MeOH=methanol

EtOH=ethanol

eq.=equivalent

Zn=zinc

TABLE 1
Reaction parameters and analysis data

| | MeCDFA + 1 eq. of | | | MeCDFA + 1.5 eq. of | | | | MeCDFA + 1.5 eq. of | | | |
|---|---|---|---|---|---|---|---|---|---|---|---|
| | Zn in MeOH, reflux | | | Zn in MeOH, 50° C. | | | | Zn in MeOH, reflux | | | |
| | 1 h | 2 h | 3 h | 1 h | 2 h | 3 h | 4 h | 1 h | 2 h | 3 h | 4 h |
| ClCF₂COOMe | 29.1¹⁾ | 17.1 | 14.3 | 72.5 | 36.8 | 12.2 | 4.9 | 43.7 | 10.9 | 1.1 | 0.3 |
| HCF₂COOMe | 58.4 | 68.8 | 70.3 | 26.8 | 59.2 | 81.1 | 84.8 | 55.9 | 88.3 | 95.6 | 95.8 |
| ClF₂COOH | 3.5 | 3.1 | 3.9 | 0.7 | 1.5 | 1.7 | 1.1 | 0.04 | 0.04 | 0 | 0 |
| HCF₂COOH | 9 | 11.1 | 11.6 | 0.3 | 2.6 | 5 | 9.1 | 0.4 | 0.8 | 3.3 | 4 |

¹⁾Data in %

It can be seen that an excess of zinc and reflux temperature of the methanol are positive for the yield.

### Example 2

### Preparation of Ethyl Difluoroacetate

The reaction was carried out as in Example 1, but in boiling ethanol. Reaction parameters and analysis data are compiled in Table 2.

TABLE 2
Reaction parameters and analysis data

| | EtCDFA + 1.5 eq. of Zn | | |
|---|---|---|---|
| | in EtOH, reflux | | |
| | 1 h | 2 h | 3 h |
| ClCF₂COOEt | 0.8¹⁾ | 0 | 0 |
| HCF₂COOEt | 61.6 | 84.9 | 89.9 |
| ClF₂COOH | 18.1 | 3.3 | 0.4 |
| HCF₂COOH | 19.5 | 11.9 | 9.7 |

¹⁾Data in %

### Example 3

### Preparation of Methyl Difluoroacetate from Methyl Chlorodifluoroacetate Prepared In Situ

Procedure: zinc was initially charged in methanol and chlorodifluoroacetyl chloride was added. The reaction proceeds exothermically with evolution of gas. Process parameters and analysis data are compiled in Table 3.

TABLE 3
Reaction parameters and analysis data

| | CDFACl + | CDFACl + | CDFACl + |
|---|---|---|---|
| | 1.5 eq. of Zn | 1.5 eq. of Zn | 1.5 eq. of Zn |
| | in MeOH, 1 h, | in MeOH, 1 h, | in MeOH, 3 h, |
| | RT | reflux | reflux |
| ClCF₂COOMe | 52.1¹⁾ | 0 | 0 |
| HCF₂COOMe | 16.7 | 24.1 | 24.1 |
| ClF₂COOH | 28.6 | 35.4 | 35.4 |
| HCF₂COOH | 2.6 | 36.1 | 36.2 |

¹⁾Data in %

The reaction appears to have ended after refluxing for 1 h. It is suspected that the HCl formed reacts very rapidly with the zinc and forms nascent hydrogen which, though, reduces the ClCF₂ group only partly. A larger excess should improve the result.

1. Process for preparing compounds having a CFₙHC(O) group from a CFₙXC(O) group and zinc in the presence of an alcohol as a proton source, where n is 1 or 2 and X is bromine, iodine or chlorine, by exchanging X for hydrogen, excluding compounds which are substituted by X both in the α-position and in the β-position.

2. Process according to claim 1, characterized in that compounds having one or more CFₙHC(O) groups are prepared from compounds having one or more CFₙClC(O) groups, where n and X are each as defined in claim 1.

3. Process according to claim 1, characterized in that an ester of the formula R¹CFHC(O)OR² is prepared, in which R¹ is F; C1-C5-alkyl; or C1-C5-alkyl which is substituted by at least 1 fluorine atom; and R² is C1-C5-alkyl; or C1-C5-alkyl which is substituted by at least 1 fluorine atom; or in that a diester of the formula R³OC(O)CFHC(O)OR³ is prepared, in which R³ is C1-C5-alkyl; or C1-C5-alkyl which is substituted by at least 1 fluorine atom.

4. Process according to claim 3, characterized in that R¹ is F or C1-C3 which is part-fluorinated or perfluorinated.

5. Process according to claim 3, characterized in that R² and R³ are each methyl, ethyl, n-propyl or isopropyl.

6. Process according to claim 3, characterized in that R¹ is F or CF₃.

7. Process according to claim 3, characterized in that the alcohol corresponds to the R² or R³ radical.

8. Process according to claim 3, characterized in that the ester is prepared in situ from acid chloride and alcohol.

9. Process according to claim 1, characterized in that the reaction product is added as a solvent.

10. The process according to claim 9, characterized in that the azeotrope of methyl difluoroacetate and methanol, which acts as a solvent and if appropriate as a proton source, is added in the preparation of methyl difluoroacetate.

11. The azeotrope of methyl difluoroacetate and methanol.

12. The process according to claim 1, wherein X is chlorine.
